(19) United States
(12) Patent Application Publication
(10) Pub. No.: US 2013/0274184 A1
(43) Pub. Date: Oct. 17, 2013

(54) ER STRESS RELIEVERS IN BETA CELL PROTECTION

(71) Applicants: NEW YORK STEM CELL FOUNDATION, (US); THE TRUSTEES OF COLUMBIA UNIVERSITY IN, (US)

(72) Inventors: Linshan Shang, New York, NY (US); Dieter Egli, New York, NY (US); Rudy Leibel, New York, NY (US)

(73) Assignees: THE TRUSTEES OF COLUMBIA UNIVERSITY IN THE CITY OF NEW YORK, New York, NY (US); NEW YORK STEM CELL FOUNDATION, New York, NY (US)

(21) Appl. No.: 13/649,040

(22) Filed: Oct. 10, 2012

Related U.S. Application Data
(60) Provisional application No. 61/545,915, filed on Oct. 11, 2011.

(51) Int. Cl. A61K 31/575; A61K 38/28; C12Q 1/02; A61K 31/192

(52) U.S. Cl. CPC A61K 31/575; A61K 31/192; A61K 38/28; C12Q 1/025
USPC 514/6.5; 514/570; 514/182; 435/29

(57) ABSTRACT

The present invention is based on the discovery that certain small molecules can relieve ER stress, leading to increased insulin production in beta cells and improved insulin secretion. Methods of treating a disease or disorder in a subject, wherein the disease or disorder is characterized by intracellular endoplasmic reticulum (ER) stress, by administering to the subject, an effective amount of a compound that is an ER stress reliever, are provided herein.

### RELATED APPLICATIONS

This application claims benefit of priority under 35 U.S.C. §119(e) to U.S. Provisional application 61/545,915 filed Oct. 11, 2011 entitled “ER Stress Relievers in Beta Cell Protection”, which is incorporated by reference in its entirety.

### FIELD OF THE INVENTION

The invention is generally directed to protein folding and more specifically to methods of treating diseases associated with endoplasmic reticulum stress (ER), including diabetes.

### BACKGROUND OF THE INVENTION

All forms of diabetes are ultimately caused by an inability of beta cells in the pancreas to provide sufficient insulin in response to ambient blood glucose concentrations. Autoimmunity in Type 1 diabetes (T1D) and peripheral insulin resistance in Type 2 diabetes (T2D) are important initiating mechanisms, but may not be the only factors resulting in reductions of beta cell functionality and mass. In T1D, autoimmunity precedes diabetes for several years, and beta cells are still present more than 8 years after diagnosis, but these residual beta cells are functionally compromised. During development of T2D, beta cells may initially compensate for peripheral insulin resistance by increasing insulin production and beta cell mass, but eventually fail in both; at advanced stages, beta cell mass and functionality is greatly reduced. Diabetes can also be caused by mutations in genes involved in beta cell function, causing maturity onset diabetes of the young (MODY), such as mutations in GCK (glucokinase), KCNJ11 (a potassium channel), or WFS1 (Wolfram syndrome).

Diabetes mellitus is a serious metabolic disease that is defined by the presence of chemically elevated levels of blood glucose (hyperglycemia). The term diabetes mellitus encompasses several different hyperglycemic states. These states include Type 1 (insulin-dependent diabetes mellitus or IDDM) and Type 2 (non-insulin dependent diabetes mellitus or NIDDM) diabetes. The hyperglycemia present in individuals with Type 1 diabetes is associated with deficient, reduced, or nonexistent levels of insulin that are insufficient to maintain blood glucose levels within the physiological range. Conventionally, Type 1 diabetes is treated by administration of replacement doses of insulin, generally by a parenteral route.

Type 2 diabetes is an increasingly prevalent disease of aging. It is initially characterized by decreased sensitivity to insulin and a compensatory elevation in circulating insulin concentrations, the latter of which is required to maintain normal blood glucose levels.

Wolfram syndrome is characterized by juvenile-onset diabetes, optic atrophy, deafness and neurological degeneration. The disease is fatal and no treatments for the diabetes other than provision of exogenous insulin are available. Wolfram syndrome is caused by mutations in WFS1 gene, which is highly expressed in human islets. Postmortem analysis of pancreata of Wolfram subjects showed a selective loss of pancreatic beta cells. In the mouse, loss of the WFS1 gene results in impaired glucose-stimulated insulin secretion, upregulation of ER stress markers, reduced insulin content, and a selective loss of beta cells in pancreatic islets. How dysfunctional WFS1 causes these phenotypes is not clear. WFS1 deficiency was reported to reduce insulin processing and acidification in insulin granules of mouse beta cells, where low pH is necessary for insulin processing and granule exocytosis. In cultured human cells, ectopically expressed WFS1 localizes to the endoplasmic reticulum (ER), where it physically interacts with calmodulin in a Ca2+-dependent manner and modulates free Ca2+ homeostasis, which is crucial for protein folding and insulin exocytosis. WFS1-deficient mouse islets showed reduced glucose-stimulated rise in the cytosolic calcium. In mouse islets, following stimulation with high concentrations of glucose, WFS1 can also be found on the plasma membrane, where it interacts with adenylyl cyclase and stimulates cAMP synthesis, thereby promoting insulin secretion. In addition, WFS1 deficiency leads to the activation of the unfolded protein response (UPR) components, such as GRP78 (Bip) and XBP-1 and decreases the ubiquitination of ATF6α. The unfolded protein response coordinates protein-folding capacity with transcriptional regulation and protein synthesis to mitigate ER stress. The UPR may be particularly important for beta cells, which have obligate high levels of protein production and secretion. Failure to resolve unfolded protein response results in persistent decreases in translation and a loss of cellular functionality, or in cell death by apoptosis.

The endoplasmic reticulum (ER) is a cellular compartment responsible for multiple important cellular functions including the biosynthesis and folding of newly synthesized proteins destined for secretion, such as insulin. A myriad of pathological and physiological factors perturb ER function and cause dysregulation of ER homeostasis, leading to ER stress. ER stress elicits a signaling cascade to mitigate stress, the unfolded protein response (UPR). As long as the UPR can relieve stress, cells can produce the proper amount of proteins and maintain ER homeostasis. If the UPR, however, fails to maintain ER homeostasis, cells will undergo apoptosis. Activation of the UPR is critical to the survival of insulin-producing pancreatic beta-cells with high secretory protein production. Any disruption of ER homeostasis in beta-cells can lead to cell death and contribute to the pathogenesis of diabetes.

### SUMMARY OF THE INVENTION

The present invention is based on the seminal discovery that certain small molecules can relieve ER stress, leading to increased insulin production in beta cells and improved insulin secretion. While not wanting to be bound by a particular theory, it is believed that the present invention methods may lead to increased beta cell survival as well. Using a cellular model of diabetes based on patient-derived induced pluripotent stem cells (iPSCs), it was found that beta cells derived from WFS1 mutant stem cells showed insulin processing and insulin secretion in response to various secretagogues comparable to healthy controls, but had lower total insulin content and increased activity of unfolded protein response (UPR) pathways. Importantly, the chemical chaperone 4-phenylbutyric Acid (PBA) reduced the activity of UPR pathways, and restored normal insulin content. In contrast, experimental ER stress further reduced insulin content, impaired insulin processing and abolished stimulated insulin secretion in Wolfram beta cells, while cells from controls remained unaffected. PBA protected beta cells from these detrimental effects of ER stress. These results show that ER stress plays a central role in beta cell dysfunction, and demonstrate that beta cell function can be improved using chemical chaperones.

In one embodiment, the invention provides a method of treating a disease or disorder in a subject, wherein the disease or disorder is characterized by intracellular endoplasmic reticulum (ER) stress, comprising administering to the subject, an effective amount of a compound that is an ER stress reliever, thereby treating the disease or disorder. In one aspect, the compound is 4-phenylbutyric acid (PBA) or Tauroursodeoxycholic acid (TUDCA). In a further aspect, the disease or disorder is diabetes (type 1 or type 2), Wolcott-Rallison syndrome, Permanent neonatal Diabetes, PERK−/− (global elevation or ER stress) or Wolfram syndrome.

In yet another embodiment, the invention provides a method of inhibiting beta cell loss in a subject with diabetes (type 1 or type 2), comprising administering to the subject, an effective amount of an ER stress reliever compound, thereby inhibiting beta cell loss in the subject. In one aspect, the compound is a small molecule. In certain aspects, the compound is 4-phenylbutyric Acid (PBA) or Tauroursodeoxychlic Acid (TUDCA).

In another aspect, the invention methods include further administering exogenous insulin to the subject. The subject can be any mammal, preferably a human.

In another embodiment, the invention provides a method of identifying a compound that is an ER stress reliever comprising contacting a beta cell, in vitro or in vivo, with a test compound and measuring the level of insulin produced or protein folding prior to and following contacting with the test compound, wherein an increase in insulin levels or alteration in protein folding after contacting is indicative of an ER stress reliever compound. In one aspect, the beta cell is derived from a subject having diabetes. The beta cells can be derived from a pluripotent stem cells of a subject with diabetes. Such pluripotent stem cells can be obtained by a number of methods such as the illustrative method shown herein, which is by iPSC. Other methods are well known in the art.

### DESCRIPTION OF THE DRAWINGS

FIG. 1 shows that induced pluripotent stem cells (iPSCs) from Wolfram subjects were efficiently differentiated into insulin-producing cells. FIG. 1A is a diagram of WFS1 structure showing the mutation sites and Sanger sequencing profiles in the 4 Wolfram subjects described herein. Arrows indicate the four deleted nucleotides (CTCT). FIG. 1B shows immunostaining of Wolfram cultures differentiated to endoderm (SOX17), pancreatic endoderm (PDX1) and C-peptide positive cells. FIG. 1C shows the differentiation efficiency in control and WFS1 cells using imaging. N=10 for each of 3 independent experiments. FIG. 1D is a representative FACS showing percentage of C-peptide positive cells in differentiated control and WFS1 cells. FIG. 1E shows immunostaining analysis of WFS1, glucagon and C-peptide in iPS-derived pancreatic Wolfram cell cultures.

FIG. 2 shows that reduced insulin production in Wolfram beta cells can be rescued by ER stress reliever 4PBA. FIG. 2A shows insulin mRNA levels in control and WFS1 beta cells normalized to TBP mRNA levels and to the number of insulin positive cells used for analysis. FIG. 2B shows insulin protein content in control and WFS1 beta cells under indicated conditions. Error bars represents 3 independent experiments with three replicates in each experiment. FIG. 2C shows transmission electron microscope (TEM) images of representative control and WFS1 cells. Scale bar is 2 nm. FIG. 2D shows the quantification of granule numbers per section of control and WFS1 cells. Two independent experiments with n=9 sections for each subject of each experiment. FIG. 2E shows the fold change of spliced XBP-1 mRNA levels in control and Wolfram beta cell cultures treated with vehicle or 4PBA for 7 days. FIG. 2F shows the fold change of GRP78 mRNA level in control and Wolfram iPS cells at increasing concentration of TG treatment for 6 hours. * P<0.05. FIG. 2G shows the fold change of GRP78 mRNA levels in Wolfram iPSCs upon different treatments. * P<0.05. TG: thapsigargin; 10 nM. 4PBA: Sodium 4-phenylbutyrate; 1 mM. TUDCA: tauroursodeoxycholate; 1 mM. FIG. 2H shows representative TEM images showing endoplasmic reticulum morphology in control and WFS1 cells after 12 hours treatment of 10 nM TG. Arrows point to ER structure. Scale bar is 500 nm.

FIG. 3 shows that insulin secretion function and insulin processing are more vulnerable to ER stress. FIG. 3A shows the fold change of human C-peptide secretion in response to indicated secretagogues. Cells were treated with 5.6 mM glucose for 1 hour followed by 16.9 mM glucose, or 15 mM arginine, or 30 mM potassium, or 1 mM DBcAMP+16.9 mM glucose. Results present three independent experiments with n=3 for each experiment. * P<0.05 of TG vs. Vehicle; # P<0.05 of TG+4PBA vs. TG. FIG. 3B shows the fold change of human C-peptide secretion to glucose stimulation calculated as amount of C-peptide secreted in response to 16.9 mM glucose divided by C-peptide secreted in response to 5.6 mM glucose. N=3 for each of two independent experiments. FIG. 3C shows the Proinsulin/insulin ratio in control and WFS1 cells under indicated conditions. N=6 for each of two independent experiments. FIG. 3D shows the fold change of human C-peptide and glucagon in control and WFS1 cells under indicated conditions. N=3 for each experiment of 3 independent experiments. TG: thapsigargin; 10 nM, 12 hour treatment. 4PBA: Sodium 4-phenylbutyrate; 1 mM, 1 hour treatment prior to and 12 hour during TG treatment.

FIG. 4 shows that Wolfram beta cells showed reduced glucose response in vivo. FIG. 4A shows human C-peptide level in the sera of recipient and negative control mice before and after nephrectomy. FIG. 4B shows basal human C-peptide level in the sera of mice transplanted with human islets, control and WFS1 cells. FIG. 4C shows the fold change of human C-peptide in the sera of mice transplanted with human islets, control and WFS1 cells before and 30 mins after glucose (1 mg/g body weight) IP injection. FIG. 4D shows the fold change of human C-peptide levels (before and after glucose injection) produced by human islets and WFS1 implants during 90 day period. FIG. 4E shows immunohistochemistry analysis of transplanted control and WFS1 beta cells. Representative images showing human C-peptide and ATF6α positive cells in transplants.

FIG. 5 shows that induced pluripotent stem (iPS) cells generated from Wolfram fibroblasts using Sendai virus vectors. FIG. 5A. Wolfram subject fibroblasts and Wolfram subject iPS cells. FIG. 5B. Karyotypes of the iPS cells of four Wolfram research subjects. FIG. 5C. The Wolfram iPS cells expressed pluripotent marker genes, shown are SSEA4, SOX2, TRA-1-60, NANOG, TRA-1-81, OCT4, by immunocytochemistry. FIG. 5D shows immunohistochemistry of embryonic body cultures and histological analysis of teratomas derived from iPS cells.

FIG. 6 shows enhanced unfolded protein response in Wolfram cells. FIG. 6A. Basal GRP78 mRNA levels in Control and Wolfram iPS cells. Quantification represents the results from studies of 4 Wolfram subject lines of three independent experiments. FIG. 6B. Gel image showing splicing of XBP-1 mRNA level in control and Wolfram iPS cells under indicated conditions and quantification represents the results from studies of 4 Wolfram subject lines of three independent experiments. FIG. 6C. Western blot analysis showing GRP78 expression level in control and Wolfram fibroblasts under indicated conditions. Quantification represents the results from studies from 2 Wolfram subjects (WS-1 and WS-2) of three independent experiments. TM: tunicamycin; 4PBA: Sodium 4-phenylbutyrate.

FIG. 7 shows insulin secretion of Wolfram beta cells derived from Wolfram iPSCs generated by using retrovirus vectors, instead of Sendai virus. FIG. 7A. Fold change of human C-peptide secretion to 16.9 mM glucose stimulation in control and Wolfram beta cells. N=3 for each experiment of three independent experiments. FIG. 7B. Expression from the retroviral transgenes in different cell lines as indicated. This shows that the viral vectors expression was silenced in the iPS cells.

FIG. 8 shows insulin secretion of Wolfram beta cells upon tunicamycin (TM) treatment. Fold change of human C-peptide secretion to 30 mM potassium stimulation in control and Wolfram beta cells. N=3 for each experiment of three independent experiments. 4PBA: Sodium 4-phenylbutyrate.

### DETAILED DESCRIPTION OF THE INVENTION

The present invention is based on the discovery that certain compounds are effective for improving the survival of beta cells in the pancreas. Based on the findings herein, the invention provides methods for treating diabetes and other diseases where survival of beta cells is important.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The terms “beta cell” or “pancreatic beta cell” are interchangeable as used herein and refer to cells in the pancreatic islets that are of the lineage of cells that produce insulin in response to glucose. Beta cells are found in the islets of Langerhans in the pancreas. Beta cells secrete insulin in a regulated fashion in response to blood glucose levels. In Type I or insulin dependent diabetes mellitus (IDDM) beta cells are destroyed through an auto-immune process. Since the body can no longer produce endogenous insulin, injections of exogenous insulin are required to maintain normal blood glucose levels.

As used herein, the term “treatment,” when used in the context of a therapeutic strategy to treat a disease or disorder, means any manner in which one or more of the symptoms of a disease or disorder are ameliorated or otherwise beneficially altered. As used herein, amelioration of the symptoms of a particular disease or disorder refers to any lessening, whether permanent or temporary, lasting or transient that can be attributed to or associated with treatment by the compositions and methods of the present invention (e.g., promotion of beta cell survival; increased insulin production in a subject).

The terms “effective amount” and “effective to treat,” as used herein, refer to an amount or a concentration of one or more compounds or a pharmaceutical composition described herein utilized for a period of time (including in vitro and in vivo acute or chronic administration and periodic or continuous administration) that is effective within the context of its administration for causing an intended effect or physiological outcome.

Effective amounts of one or more compounds or a pharmaceutical composition for use in the present invention include amounts that promote beta cell survival or increase levels of insulin production, or a combination thereof.

The term “subject” is used throughout the specification to describe an animal, human or non-human, to whom treatment according to the methods of the present invention is provided.

The beta cells used in the invention can be derived from a pluripotent stem cells of a subject with diabetes. Such pluripotent stem cells can be obtained by a number of methods such as the illustrative method shown herein, which is by iPSC.

By “pluripotent stem cells”, it is meant cells that can a) self-renew and b) differentiate to produce all types of cells in an organism. The term “induced pluripotent stem cell” encompasses pluripotent stem cells, that, like embryonic stem (ES) cells, can be cultured over a long period of time while maintaining the ability to differentiate into all types of cells in an organism, but that, unlike ES cells (which are derived from the inner cell mass of blastocysts), are derived from somatic cells, that is, cells that had a narrower, more defined potential and that in the absence of experimental manipulation could not give rise to all types of cells in the organism. iPS cells have an hESC-like morphology, growing as flat colonies with large nucleo-cytoplasmic ratios, defined borders and prominent nuclei. In addition, iPS cells express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. In addition, the iPS cells are capable of forming teratomas. In addition, they are capable of forming or contributing to ectoderm, mesoderm, or endoderm tissues in a living organism.

In one embodiment, the invention provides a method of identifying a compound that is an ER stress reliever. The compound can be a small molecule, a nucleic acid (e.g., DNA or RNA), antisense, RNAi, peptide, polypeptide, mimetic and the like. The method includes contacting a beta cell, in vitro or in vivo, with a test compound and measuring the level of insulin produced prior to and following contacting with the test compound, wherein an increase in insulin levels after contacting is indicative of an ER stress reliever compound. In one aspect, the beta cell is derived from a subject having diabetes. In a particular aspect, the beta cell is derived from a pluripotent stem cell of a subject having diabetes. The beta cell can be derived from differentiation of a pluripotent stem cell, for example, using iPSC.

The beta cells of the invention can be derived by various methods using for example, adult stem cells, embryonic stem cells (ESCs), epiblast stem cells (EpiSCs), and/or induced pluripotent stem cells (iPSCs; somatic cells that have been reprogrammed to a pluripotent state). Illustrative iPSCs are stem cells of adult origin into which the genes Oct-4, Sox-2, c-Myc, and Klf have been transduced, as described by Takahashi and Yamanaka (Cell 126(4):663-76 (2006)). Other exemplary iPSC's are adult stem cells into which OCT4, SOX2, NANOG, and LIN28 have been transduced (Yu, et al., Science 318:1917-1920 (2007)). One of skill in the art would know that a cocktail of reprogramming factors could be used to produce iPSCs such as factors selected from the group consisting of OCT4, SOX2, KLF4, MYC, Nanog, and Lin28. Further, the methods described herein for producing iPSCs are illustrative of the method of the present invention for deriving beta cells.

Differentiation of pluripotent stem cells may be monitored by a variety of methods known in the art. Changes in a parameter between a stem cell and a differentiation factor-treated cell may indicate that the treated cell has differentiated. Microscopy may be used to directly monitor morphology of the cells during differentiation. As an example, the differentiating pancreatic cells may form into aggregates or clusters of cells. The aggregates/clusters may contain as few as 10 cells or as many as several hundred cells. The aggregated cells may be grown in suspension or as attached cells in the pancreatic cultures.

Changes in gene expression may also indicate beta cell differentiation. Increased expression of beta cell-specific genes may be monitored at the level of protein by staining with antibodies. Antibodies against insulin, Glut2, Igf2, islet amyloid polypeptide (IAPP), glucagon, neurogenin 3 (ngn3), pancreatic and duodenal homeobox 1 (PDX1), somatostatin, c-peptide, and islet-1 may be used. Cells may be fixed and immunostained using methods well known in the art. For example, a primary antibody may be labeled with a fluorophore or chromophore for direct detection. Alternatively, a primary antibody may be detected with a secondary antibody that is labeled with a fluorophore, or chromophore, or is linked to an enzyme. The fluorophore may be fluorescein, FITC, rhodamine, Texas Red, Cy-3, Cy-5, Cy-5.5. Alexa.sup.488, Alexa.sup.594, QuantumDot.sup.525, QuantumDot.sup.565, or QuantumDot.sup.653. The enzyme linked to the secondary antibody may be HRP, beta-galactosidase, or luciferase. The labeled cell may be examined under a light microscope, a fluorescence microscope, or a confocal microscope. The fluorescence or absorbance of the cell or cell medium may be measured in a fluorometer or spectrophotomer.

Changes in gene expression may also be monitored at the level of messenger RNA (mRNA) using RT-PCR or quantitative real time PCR. RNA may be isolated from cells using methods known in the art, and the desired gene product may be amplified using PCR conditions and parameters well known in the art. Gene products that may be amplified include insulin, insulin-2, Glut2, Igf2, LAPP, glucagon, ngn3, PDX1, somatostatin, ipf1, and islet-1. Changes in the relative levels of gene expression may be determined using standard methods. The expression of alpha-, beta-, gamma-, and delta-cell specific markers may show that the cell populations are composed of all four distinct types and three major types of pancreatic cells.

The compounds of the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous use). Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

When employed as pharmaceuticals, the sulfonamide derivatives of this invention are typically administered in the form of a pharmaceutical composition. Such compositions can be prepared in a manner well known in the pharmaceutical art and comprise at least one active compound. Generally, the compounds of this invention are administered in a pharmaceutically effective amount. The amount of the compound actually administered will typically be determined by a physician in the light of the relevant circumstances, including the condition to be treated, the chosen route of administration, the actual compound administered, the age, weight, and response of the individual patient, the severity of the patient's symptoms, and the like.

The pharmaceutical compositions of these inventions can be administered by a variety of routes including oral, rectal, transdermal, subcutaneous, intravenous, intramuscular, intrathecal, intraperitoneal and intranasal. Depending on the intended route of delivery, the compounds are preferably formulated as either injectable, topical or oral compositions. The compositions for oral administration may take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the sulfonamide compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatine; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As above mentioned, the sulfonamide derivatives of formula I in such compositions is typically a minor component, frequently ranging between 0.05 to 10% by weight with the remainder being the injectable carrier and the like.

The above described components for orally administered or injectable compositions are merely representative. Further materials as well as processing techniques and the like are set out in Part 5 of Remington's Pharmaceutical Sciences, 20.sup.th Edition, 2000, Marck Publishing Company, Easton, Pa., which is incorporated herein by reference.

The compounds of this invention can also be administered in sustained release forms or from sustained release drug delivery systems. A description of representative sustained release materials can also be found in the incorporated materials in Remington's Pharmaceutical Sciences.

The compounds of the invention can be co-administered with insulin, either prior to, simultaneously with or following administration of invention compounds. Insulin is a polypeptide composed of 51 amino acids which are divided between two amino acid chains: the A chain, with 21 amino acids, and the B chain, with 30 amino acids. The chains are linked together by two disulfide bridges. Insulin preparations have been employed for many years in diabetes therapy. Such preparations use not only naturally occurring insulins but also, more recently, insulin derivatives and insulin analogs.

Insulin analogs are analogs of naturally occurring insulins, namely human insulin or animal insulins, which differ by replacement of at least one naturally occurring amino acid residue by other amino acids and/or by addition/deletion of at least one amino acid residue, from the corresponding, otherwise identical, naturally occurring insulin. The amino acids in question may also be amino acids which do not occur naturally.

Insulin derivatives are derivatives of naturally occurring insulin or an insulin analog which are obtained by chemical modification. The chemical modification may consist, for example, in the addition of one or more defined chemical groups to one or more amino acids. Generally speaking, the activity of insulin derivatives and insulin analogs is somewhat altered as compared with human insulin.

The invention is further elaborated with the help of following examples. However, these examples should not be construed to limit the scope of the invention.

### Example 1

Methods

Research Subjects and Cell Lines

Skin biopsies from subjects WS-1 and WS-2 were obtained at the Naomi Berrie Diabetes Center (New York), using an AcuPunch biopsy kit (Acuderm Inc). Fibroblast cells from WS-3, WS-4 and carrier were obtained from Coriell Research Institute (New Jersey), with the respective product number of GM01610, GM01611 and GM01701. All human subjects research was approved by the Columbia IRB and ESCRO committees. Research subjects signed informed consent and samples were coded. Skin biopsies were cut into 10-12 small pieces, and every 2-3 pieces were placed under a glass cover slip in a well of a six-well dish. The cover slips were adhered to the bottom of the culture dish by silicon droplets. 5 ml of biopsy plating media were added into each well. 5 days later, culture medium was used to replace the plating medium. Biopsy pieces were grown in culture medium for 3-4 weeks, with medium changes twice weekly. Biopsy plating medium contained DMEM, FBS, GlutaMAX, Anti-Anti, NEAA, 2-Mercaptoethanol and nucleosides and culture medium was composed of DMEM, FBS, GlutaMAX and Pen-Strep (all from Invitrogen).

Generation of Induced Pluripotent Stem Cells

Induced pluripotent stem cells were generated from fibroblast cells using the CytoTune™-iPS Sendai Reprogramming Kit (Invitrogen). 50,000 fibroblast cells were seeded in a well of six-well dish at passage three in fibroblast medium. Next day, Sendai viruses expressing human transcription factors Oct4, Sox2, Klf4 and C-Myc were mixed in fibroblast medium to infect fibroblast cells according to the manufacturer's instructions, 2 days later, the medium was exchanged to human ES medium supplemented by the MEK inhibitor PD0325901 (0.5 μM; Stemgent), ALK5 inhibitor SB431542 (2 μM; Stemgent), and thiazovivin (0.5 μM; Stemgent). Alternatively, iPS cells were generated with retroviral vectors (Takahashi, Tanabe et al. 2007) and tested for transgene inactivation by RT-PCR. Human ES medium contained the following: KO-DMEM, KSR, GlutaMAX, NEAA, 2-Mercaptoethanol, PenStrep and bFGF (all from Invitrogen). Individual colonies of induced pluripotent stem cells were recognized based on morphology and picked between day 21-28 post infection. Each iPS cell line was expanded from a single colony. All iPS cells lines were cultured on feeder cells with human ES medium. Karyotyping of the cells was performed by Cell Line Genetics Inc. (Wisconsin). To generate embryoid bodies, 1-2 million iPS cells of each line were detached by TrypLE (Invitrogen) treatment; cells were then collected and cultured into a low-attachment 6-well culture dish with human ES medium containing 10 μM ROCK inhibitor (Y27632). The next day, medium was changed to fibroblast culture medium and keep culturing for 3 weeks. Cells formed sphere morphology and were collected for immunostaining analysis. For teratoma analysis, 1-2 million cells of each iPS cell line were detached and collected by TrypLE treatment. Cells were suspended in 0.5 ml of human ES medium and mixed with 0.5 ml matrigel (BD Biosciences) and injected subcutaneously into dorsal flanks of a NOD.Cg-Prkdcscid Il2rgtm1Wjl/SzJ (NSG) mouse (Stock No. 005557, The Jackson Laboratory). 8-12 weeks after injection, teratomas were collected, fixed overnight with 4% paraformaldehyde and processed for paraffin embedding according to standard procedures. Then the samples were sectioned and HE (hematoxylin and eosin) stained.

Beta Cells Differentiation

Human ES or iPS cells were dissociated by Dispase (3-5 mins) and Accutase (5 mins, Sigma). Cells were suspended in human ES medium containing 10 μM Y27632, a ROCK inhibitor, and filtered through a 70 μm cell strainer. Then cells were seeded at a density of 800,000 cells/well in 12-well plates. After 1 or 2 days, when cells reached 80-90% confluence, differentiation was started. On Day 1: cells were briefly washed once with RPMI medium, then were treated with Activin A (100 ng/ml), Wnt3A (25 ng/ml) and 0.075 mM EGTA in RPMI medium. On day 2-3: cells were treated with Activin A (100 ng/ml) and 0.2% FBS in RPMI medium. On day 4-5: cells were treated with FGF10 (50 ng/ml), KAAD-cyclopamine (0.25 μM) and 2% FBS in RPMI medium. On day 6-8: cells were treated with FGF10 (50 ng/ml), KAAD-cyclopamine (0.25 μM), retinoic acid (2 μM) and LDN-193189 (250 nM), B27 in DMEM medium. On day 9-10: cells were treated with exendin-4 (50 ng/ml), SB431542 (2 μM) and B27 in CMRL medium. On day 11-12, cells were treated with T4 (thyroid hormone, 0.02 nM) and B27 in CMRL medium. After day 12, cells were incubated in CMRL medium with B27. Cells were analyzed between day 14 and day 16.

Immunostaining

Cells were washed once with PBS and then fixed by 4% paraformaldehyde for 30 minutes at room temperature. Embryoid bodies and mouse kidneys were fixed with 4% paraformaldehyde overnight at 4° C., dehydrated using 15% (w/v) sucrose and 30% (w/v) sucrose solution and embedded in OCT compound (Tissue-Tek), and then frozen under −80° C. Cells or sections were blocked in 5% normal donkey serum for 30 minutes at room temperature. Primary antibodies used in the study were as follows: mouse-anti-SSEA4 (MAB1435; R&D systems), rabbit-anti-SOX2 (09-0024; stemgent), mouse-anti-TRA1-60 (MAB4360; Millipore), goat-anti-NANOG (AF1997; R&D systems), mouse-anti-TRA1-81 (MAB4381; Millipore), mouse-anti-OCT4 (sc-5279; Santa Cruz Biotechnology), rabbit-anti-AFP (A000829; DAKO), mouse-anti-SMA (A7607; Sigma), rabbit-anti-TUJ1 (T3952; Sigma), goat-anti-SOX17 (AF1924; R&D systems), goat-anti-PDX1 (AF2419; R&D systems), mouse-anti-C-peptide (05-1109; Millipore), rabbit-anti-glucagon (A056501; DAKO). Anti WFS1 antibody was generously provided by Dr. Urano, Fumihiko. Second antibodies were obtained from Molecular Probes (Invitrogen). Cell images were acquired by using an Olympus 1×71 fluorescence microscope and confocal microscope (ZEISS).

Unfolded Protein Response (UPR) Analysis

Wolfram and control iPSCs or fibroblasts were incubated with indicated dosages of thapsigargin (TG) or tunicamycin (TM) (Both were from Sigma) for 6 hours after an overnight starvation. 1 mM Sodium 4-phenylbutyrate (4PBA) (EMD Chemicals Inc.) was administrated one hour prior to and through TG or TM treatment. Cells were harvested and subjected to RNA and protein analysis. In vitro differentiated beta cells were treated with 10 nM TG for 12 hours, or 0.5 μg/ml TM for 6 hours with or without 1 mM 4PBA treatment one hour prior to and through TG or TM treatment. For long-term 4PBA treatment, cells were incubated with 1 mM 4PBA starting on day 9 of differentiation, when cells reached pancreatic endoderm stage, and maintained until day 15. Then cells were subjected to insulin secretion, RNA and protein analysis. RNA was isolated using RNAeasy plus kit (Qiagen). cDNA was generated by using RT kit (Promega). Primers for PCR analysis were as follows: XBP-1 for gel-imaging (Lee, Won et al.) forward 5′ GAAGCCAAGGGGAATGAAGT 3′ (SEQ ID NO:1), reverse 5′ GGGAAGGGCATTTGAAGAAC 3′ (SEQ ID NO:2); sXBP-1 for QPCR (Merquiol, Uzi et al. 2011) forward 5′ CTGAGTCCGCAGCAGGTG 3′(SEQ ID NO:3), reverse 5′ TGCCCAACAGGATATCAGACT 3′ (SEQ ID NO:4); GRP78 forward 5′ CACAGTGGTGCCTACCAAGA 3′(SEQ ID NO:5), reverse 5′ TGATTGTCTTTTGTCAGGGGT 3′ (SEQ ID NO:6); Insulin forward 5′ TTCTACACACCCAAGACCCG 3′(SEQ ID NO:7), reverse 5′ CAATGCCACGCTTCTGC 3′(SEQ ID NO:8). GRP78 protein level was determined by western blot using mouse-anti GRP78 antibody (Santa Cruz, sc-166490).

Insulin and Proinsulin Content Measurement

To determine Insulin or proinsulin content within the cell, differentiated cells were collected and lysed by M-PER protein extraction reagent (Thermo Scientific). Proinsulin and insulin contents were measured by using human proinsulin and insulin ELISA kits (Mercodia). Quantification of positively stained cells was analyzed using Celigo Cytometer system (Cyntellect), and flow cytometry analysis. To normalize insulin content to beta cell number, cultures were dissociated to single cells, and divided into three fractions: 20% of cells for cell number quantification, 40% for RNA analysis and 40% for ELISA assay to determine insulin content.

In Vitro Insulin and Glucagon Secretion Assay

Cells were cultured in 12-well dishes. After 14 days of differentiation, cells were washed for 1 hour in CMRL medium, then incubated in 300 μl CMRL medium containing 5.6 mM glucose for 1 hour and the medium was collected. After that, 300 μl CMRL medium containing 16.9 mM glucose, or 15 mM arginine, or 30 mM potassium, or 1 mM DBcAMP+16.9 mM glucose was used to treat cells for 1 hour and then the medium was collected. Human C-peptide concentration in the medium was measured by ultra-sensitive human C-peptide ELISA kit according to manufacturer's instructions (Mercodia). Glucagon levels in medium were measured by using Glucagon ELISA kit (ALPCO Diagnostics).

Transmission Electron Microscopy

Differentiated beta cells were treated with or without 10 nM TG for 12 hours, and then fixed in 2.5% glutaraldehyde in 0.1 M Sorenson's buffer (pH 7.2) for one hour. Samples were processed and imaged by Dignostic Service, Department of Pathology and Cell Biology, Columbia University. Secretory granule structure and endoplasmic reticulum (ER) morphology were visually recognized. The number of granules was determined using ImageJ software.

Transplantation and IPGTT

At 14 days of differentiation, cells were dissociated using TrypLE for 3 minutes at room temperature. 2-3 million cells were collected into an eppendorf tube, spun down and the supernatant was discarded. 10-15 μl matrigel (BD Biosciences) was mixed with the cell pellet, before transplanted into kidney capsule of a NOD.Cg-Prkdcscid Il2rgtm1Wjl/SzJ (NSG) mouse (Stock No. 005557, The Jackson Laboratory), following a previously described protocol (Szot, Koudria et al. 2007). Intraperitoneal glucose tolerance tests (IPGTT) were performed between 3 to 7 months after transplantation. Mice were deprived of food overnight (12-14 hours), but have water available. In the morning, blood glucose levels of the mice were measured by pricking the tail vein. Blood samples were collected by puncturing the submandibular vein, which locates at the backend of jaw. Then each mouse was weighed, intraperitoneal injected with a glucose solution (in saline, 1 mg/g body weight). Half an hour later, the mice were analyzed for blood glucose level and blood samples were collected again. Serum was obtained by centrifuging blood samples at 4000 rpm for 15 min. And human C-peptide concentration in the mouse serum was measured by using ultra-sensitive human C-peptide ELISA kit according to manufacturer's instructions (Mercodia). Alive nephrectomy was performed on a sub-group of receipt mice after human C-peptide was detected in the mouse serum.

### Example 2

Wolfram iPS Cells Differentiate Normally into Beta Cells

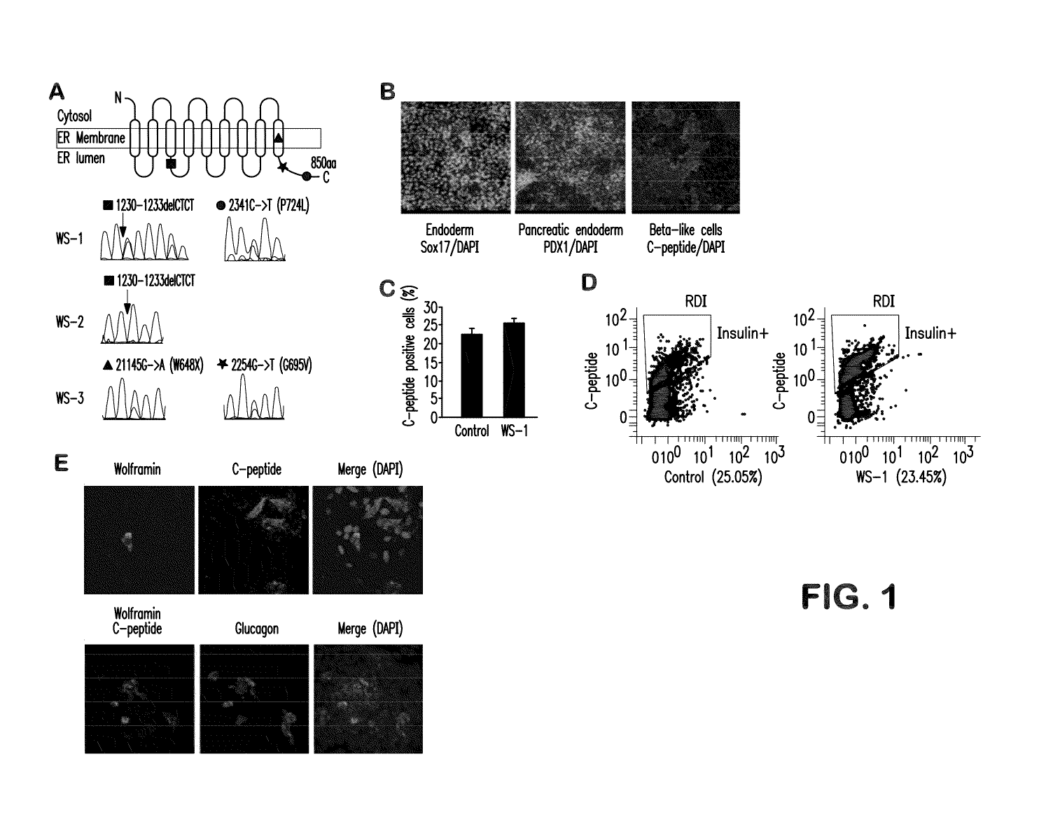

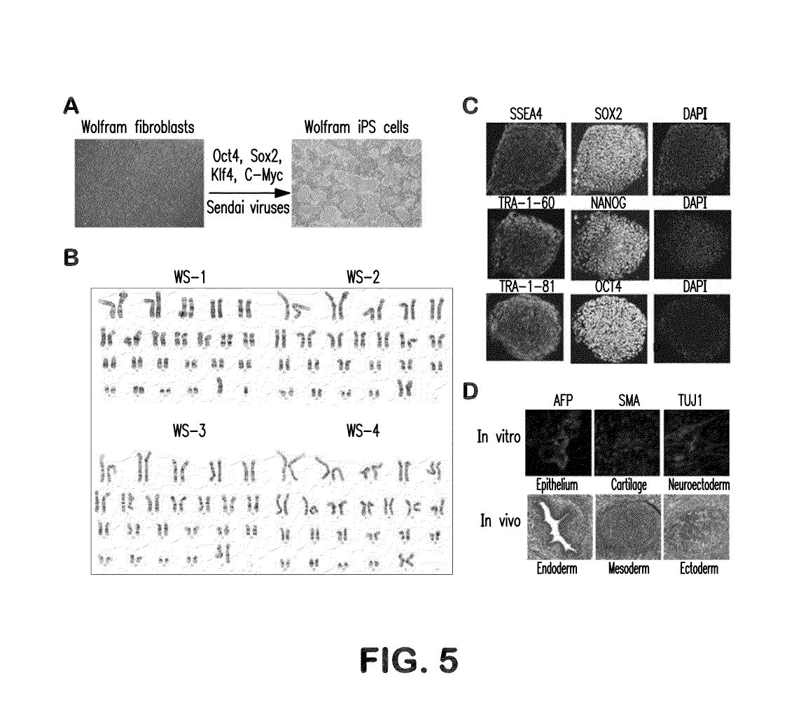

We obtained skin biopsies and established skin cell lines from two subjects affected with Wolfram syndrome, denoted: WS-1 and WS-2. Sequencing of the WFS1 locus revealed that WS-2 is homozygous for a frameshift mutation 1230-1233delCTCT (V412fsX440) (Colosimo, Guida et al. 2003), and that WS-1 is heterozygous for V412fsX440, and also carries a missense mutation P724L (Inoue, Tanizawa et al. 1998). An additional three skin cell lines were obtained from Coriell Research Institute from two siblings with Wolfram syndrome: WS-3 and WS-4, and an unaffected parent. Both WS-3 and WS-4 are heterozygous for the missense mutations W648X and G695V in the WFS1 protein (Inoue, Tanizawa et al. 1998) (FIG. 1A). All Wolfram subjects were insulin-dependent and affected by optic atrophy (Table 1). We generated induced pluripotent stem cells (iPSCs) from fibroblast cell lines using non-integrating Sendai virus vectors encoding the transcription factors Oct4, Sox2, Klf4 and c-Myc (FIG. 5A) (Fusaki, Ban et al. 2009). All iPS cell lines were karyotypically normal (FIG. 5B), expressed markers of pluripotency (FIG. 5C), and differentiated into cell types and tissues of all three germ layers in vitro and after injection into immune-compromised mice (FIG. 5D).

iPS cell lines from Wolfram and control subjects differentiated into insulin-producing cells as previously described. Differentiation efficiency of Wolfram cells was identical to controls: after 8 days of differentiation, 81.1% of total cells expressed PDX1, a marker for pancreatic endocrine progenitors, and after 13 days of differentiation, 25.6% of total cells expressed C-peptide, as determined by imaging and FACS analysis (FIG. 1B-D). To determine the expression pattern of WFS1, we performed immunostaining for WFS1 (Wolframin), insulin and glucagon. WFS1 was specifically expressed in insulin-producing cells, but not in glucagon-positive cells present in stem cell-derived islet cells from control and Wolfram subjects (FIG. 1E). Thus, stem cell-derived pancreatic cells show the expression patterns observed in the mouse pancreas, and should therefore be appropriate to study the consequences of WFS1 mutations.

TABLE 1
Information of genotypes and phenotypes of the research subjects.
Supplementary Table 1

| | | | Age of | | |
|---|---|---|---|---|---|
| | | | onset/ | Mutations | |
| Cell Line | Source | Sex | diagnosis | in WFS1 gene | Remarks |
| WS-1 | Naomi Berrie Diabetes Center | Male | 12 | 1230-1233delCTCT | Diabetes; |
| | | | | (V412fsX440), | Optic atrophy; |
| | | | | P724L | On insulin |
| WS-2 | Naomi Berrie Diabetes Center | Female | 2 | 1230-1233delCTCT | Diabetes; |
| | | | | (V412fsX440) | Optic atrophy; |
| | | | | | On insulin |
| WS-3 | Corriell Research Institute | Female | 11 | W648X, G695V | Diabetes; |
| | (GM01610) | | | | Optic atrophy; |
| | | | | | On insulin |
| WS-4 | Corriell Research Institute | Female | 13 | W648X, G695V | Diabetes; |
| | (GM01611) | | | | Optic atrophy; |
| | | | | | On insulin |
| Carrier | Corriell Research Institute | Male | Not affected | G695V | Non-diabetic, |
| | (GM01701) | | | | Father of WS-3 and WS-4 |
| Control | Harvard Universtiy^{[1]} | Male | Not affected | Normal | Non-diabetic |
| (HUES42) | | | | | |
| Control-2 | Naomi Berrie Diabetes Center | Male | Not affected | Normal | Non-diabetic |
| (iPSC) | | | | | |

### Example 3

Activated UPR Reduces Insulin Synthesis in Wolfram Beta Cells

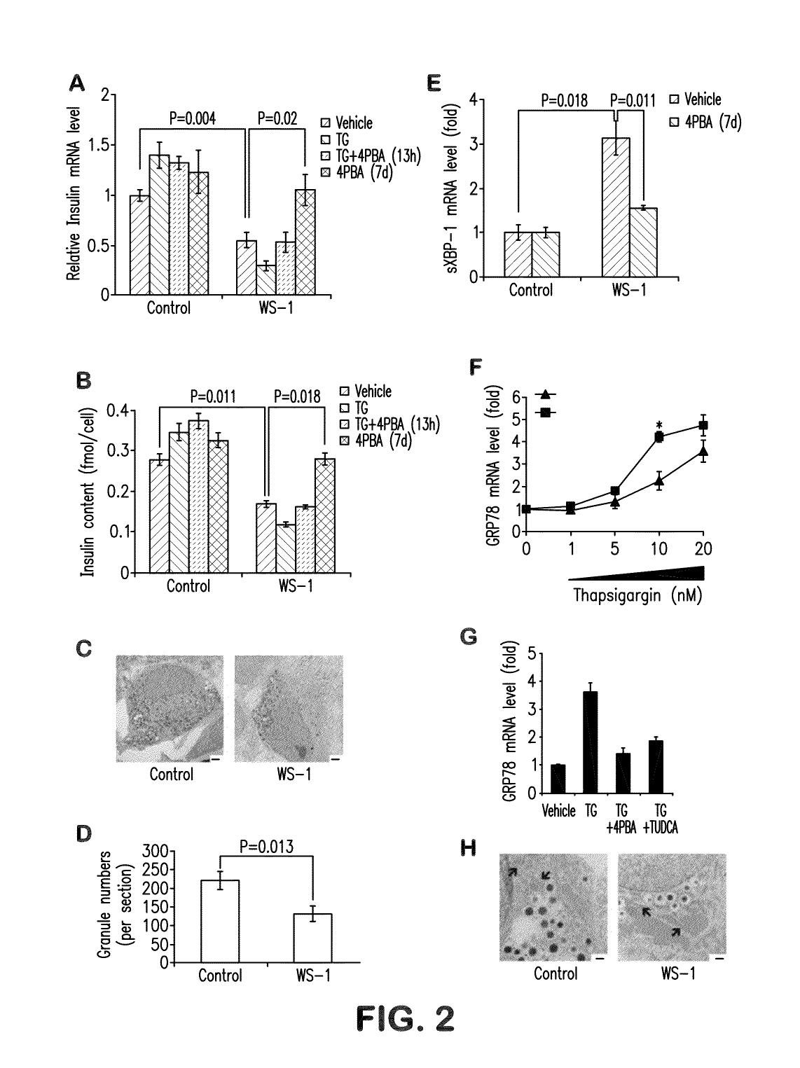

To investigate how WFS1 mutations affect beta-cell function, we first quantified insulin mRNA and protein content in Wolfram, and control stem cell-derived beta cells. To normalize insulin content to beta cell number, cultures were dissociated to single cells, and divided into three fractions to determine cell number, RNA level and insulin content. The insulin mRNA was normalized to TBP (TATA-binding protein) mRNA and to the percentage of insulin-positive cells in each sample. Similarly, insulin content was normalized to the total number of insulin-positive cells. WFS1 deficiency was associated with a 45% reduction in insulin mRNA levels compared to controls (FIG. 2A), and a 40% decrease of insulin protein content (FIG. 2B). This decrease was also reflected in the number of secretory granules imaged by transmission electron microscopy. Differentiated beta cells from unaffected individual contained abundant secretory granules. In contrast, a 41% reduction in the number of secretory granules was observed in Wolfram-derived beta cells (FIGS. 2C and D). To determine whether the lower insulin content in Wolfram beta cells was caused by increased insulin secretion, or by lower insulin synthesis, we determined the 1 hour secretion rate of C-peptide in response to 5.6 mM glucose. The rates were 0.00316 and 0.00384 fmol per hour for Wolfram and control cells, respectively. These rates are equal to 1.9% and 1.4% of insulin content in the Wolfram and control beta cells, respectively. Therefore, the reduced insulin content in Wolfram beta cells is not likely due to increased insulin secretion, but to lower rates of insulin synthesis.

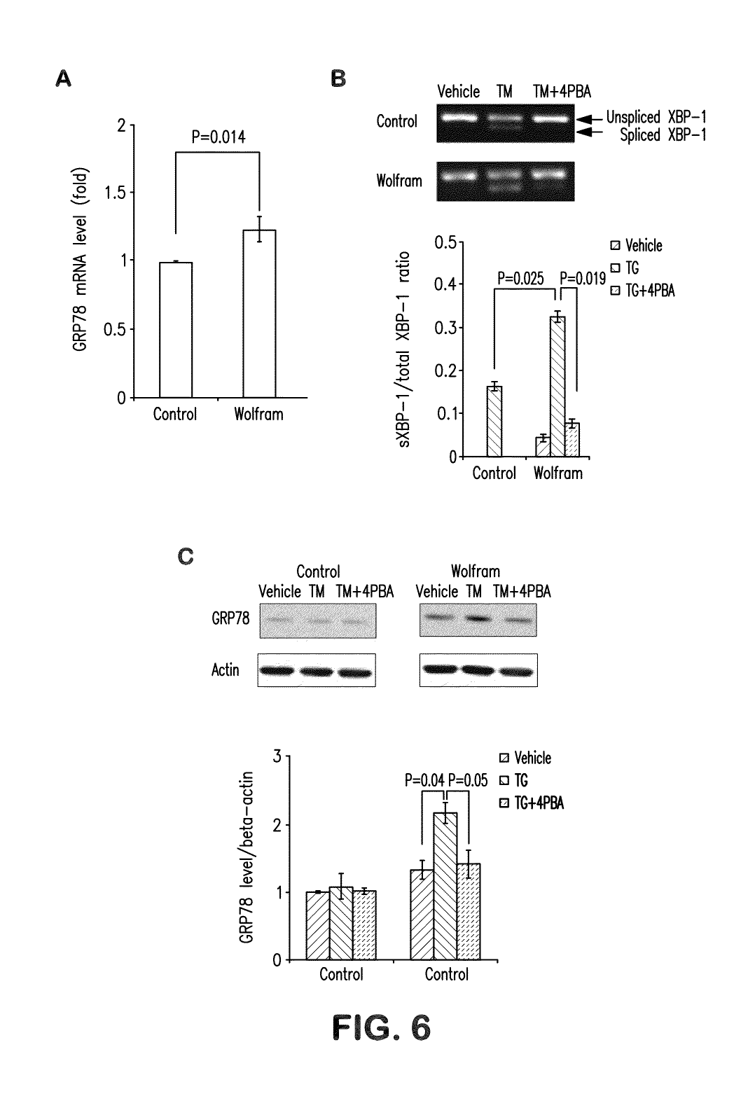

To determine the cause of the decreased insulin synthesis, we investigated the expression of components of the unfolded protein response (UPR) in Wolfram cells. IRE-1 kinase/ribonuclease and PERK, a kinase phosphorylating initiation factor 2a, sense increases in unfolded protein, and impose a state of translational repression in response to an increase in unfolded proteins. IRE-1alpha activity is reflected in the splicing of XBP-1 mRNA, allowing translation of a functional XPB-1 transcription factor (Iwawaki, Hosoda et al. 2001; Kimata, Ishiwata-Kimata et al. 2007). Long-term exposure of rat INS-1 cells to high glucose concentrations causes hyper-activation of IRE1, which leads to decreased insulin gene expression (Lipson, Fonseca et al. 2006). In beta cell cultures, iPS cells and fibroblasts, we found that levels of spliced XBP-1 mRNA, GRP78 mRNA and protein, were increased in Wolfram subject samples in comparison to controls (FIG. 2E, FIG. 6A-C). These differences between control and Wolfram cells were further enhanced by the imposition of experimental ER stress. In stem cells, thapsigargin (TG) caused a dose-dependent increase in GRP78 mRNA level and 6 hour of 10 nM TG treatment caused a greater increase of GRP78 mRNA in Wolfram cells than in control cells (4 fold versus 2 fold (FIG. 2F). Thapsigargin (TG) induces ER stress by disrupting intracellular calcium homeostasis through the inhibition of the Ca²⁺-ATPase responsible for Ca²⁺ accumulation in ER (Wong, Brostrom et al. 1993). Importantly, chemical chaperones sodium 4-phenylbutyrate (4PBA) (de Almeida, Picarote et al. 2007; Yam, Gaplovska-Kysela et al. 2007) and tauroursodeoxycholate (TUDCA) (Berger and Haller 2011) effectively reduced GRP78 mRNA levels in Wolfram cells treated with TG (FIG. 2G). Similarly, another ER stress inducer, tunicamycin (TM), which activates UPR by inhibiting N-linked glycosylation (Kozutsumi, Segal et al. 1988), induced a stronger UPR response in Wolfram iPS and fibroblast cells than in control cells. Spliced XBP-1 (sXBP-1) mRNA (FIG. 6B) and GRP78 protein levels (FIG. 6C) were higher in Wolfram cells. Both sXBP-1 and GRP78 were reduced by the addition of 4PBA.

If UPR signaling were responsible for the reduced insulin synthesis in Wolfram beta cells, elevated ER stress should further reduce insulin production, while reducing ER stress would protect insulin content. To test this inference, we experimentally increased or reduced UPR activation using TG or 4PBA in beta cell cultures. When Wolfram beta cells were generated in the presence of 4PBA from day 9 to day 15 of differentiation, sXBP-1 mRNA levels were reduced by 50% (FIG. 2E). Strikingly, this long-term incubation with 4PBA increased insulin mRNA in Wolfram cells by 1.9-fold and insulin content by 1.7-fold, to levels comparable to those in control cells without 4PBA (FIGS. 2A and B). When control cells were exposed to the same 7d treatment of 4PBA during beta-cell differentiation, a moderate increase (1.2 fold) of insulin production was also observed (FIGS. 2A and B). Exposing Wolfram beta cells to the ER stressor TG had the opposite effect: production of insulin was reduced by 46% at the mRNA level and 31% at the protein level, while control cells were unaffected (FIGS. 2A and B). Experimentally induced ER stress also affected ER morphology: the ER was greatly dilated in Wolfram beta cells in the presence of TG, while control cells remained unaffected (FIG. 2H). These results suggest that WFS1 acts in beta cells to maintain ER function under protein folding stress.

### Example 4

Normal Stimulated Insulin Secretion in WFS1 Mutant Cells

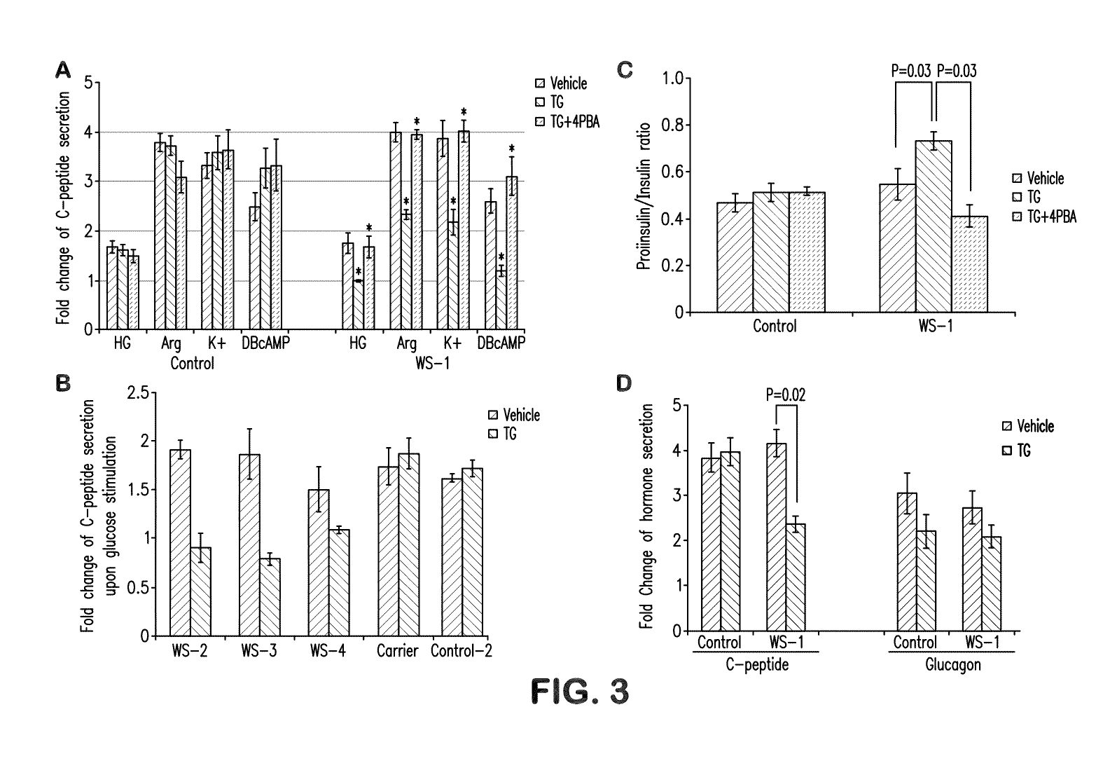

To test the ability of Wolfram beta cells to secrete insulin, we exposed them to various secretagogues, including glucose, arginine, potassium and the cAMP analog, dibutyl cAMP (DBcAMP). Our expectation was that the response to different secretagogues would reveal whether WFS1 was involved in specific steps of the cellular signals leading to insulin secretion as has been suggested by others (Fonseca, Urano et al. 2012). Glucose stimulates insulin secretion by ATP generation, resulting in the closing of the ATP sensitive potassium channel and reduction of potassium efflux, which stimulates Ca²⁺ influx and triggers exocytosis of insulin granules (Lebrun, Malaisse et al. 1982; Miki, Nagashima et al. 1998). Arginine induces insulin secretion by triggering Ca²⁺ influx, without reducing potassium efflux (Henquin and Meissner 1981; Herchuelz, Lebrun et al. 1984). cAMP influences insulin secretion by enhancing Ca⁺ influx and mobilizing insulin granules (Malaisse and Malaisse-Lagae 1984; Seino and Shibasaki 2005). And finally, extracellular potassium bypasses these upstream events by directly depolarizing the plasma membrane, resulting in the release of insulin granules (Matthews and O'Connor 1979; Matthews and Shotton 1984). To assess insulin secretion in response to glucose, we incubated cells to medium containing 5.6 mM glucose for 1 hour, followed by medium containing 16.9 mM glucose for 1 hour. Controls and heterozygous carrier beta cells showed a 1.6 to 1.7-fold higher level of C-peptide in the medium after addition of 16.9 mM glucose. A similar increase of 1.5 to 1.9 fold was seen in all four WFS1 mutant cells (FIGS. 3A and B). We further tested insulin secretion in response to arginine, potassium, and DBcAMP. Independent of the genotype and the secretagogue, a 2-4 fold increase in C-peptide secretion was observed in both control and WFS1 mutant cells (FIG. 3A). Therefore, although Wolfram beta cells showed reduced insulin content, they displayed a normal functional response to secretagogues acting at different points in metabolic sensing and insulin release.

### Example 5

Wolframin Preserves Stimulated Insulin Secretion Under Elevated ER Stress

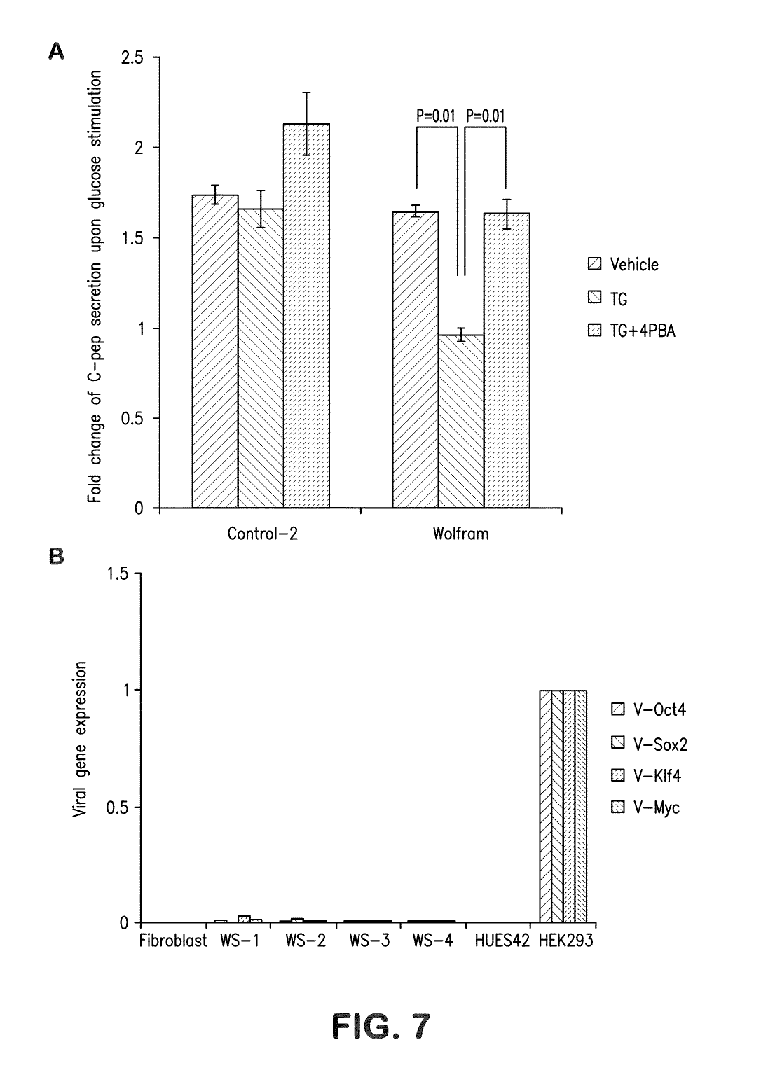

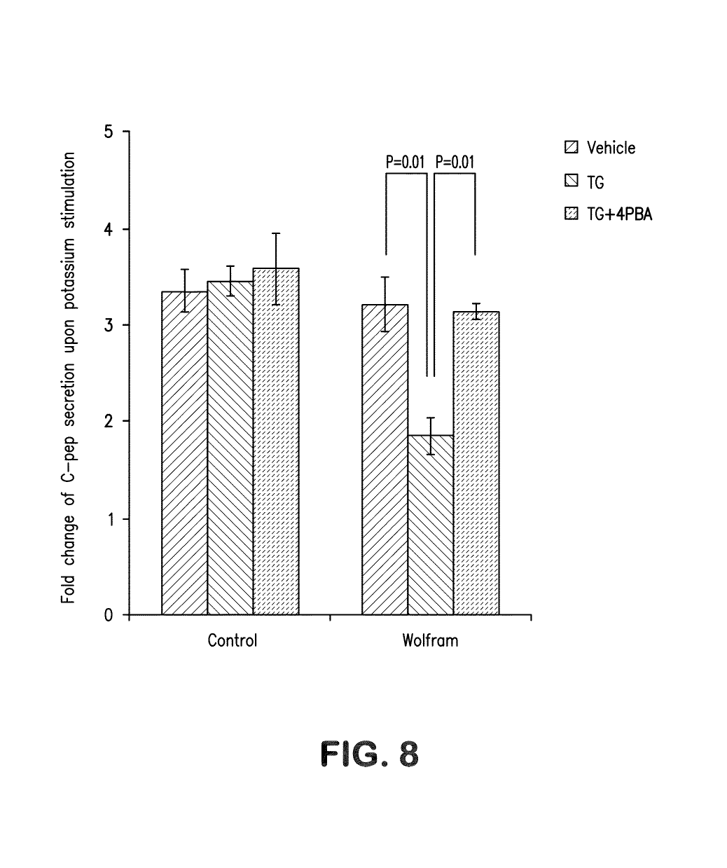

To determine whether WFS1 deficiency affected stimulated insulin secretion under ER stress, we again determined insulin secretion in response to different secretagogues. When thapsigargin (TG) treated cells were exposed to high ambient glucose (16.9 mM), Wolfram cells failed to increase insulin secretion, while control beta cells increased insulin output by 1.6 fold. Incubation with 4PBA prevented these detrimental effects of TG on Wolfram beta cells (FIG. 3A). The reduction in stimulated insulin secretion by TG was seen with all secretagogues tested, independent of their mechanism of action. When Wolfram beta cells were treated with TG, the fold increase of C-peptide in the medium decreased from 4.0 to 2.3 fold in response to arginine; and insulin-secretion in response to potassium dropped from 3.9 fold to 2.2 fold; the response to DBcAMP declined from 2.6 to 1.2 fold. Independent of the secretagogue used for stimulation, 4PBA prevented the decrease in insulin secretion upon application of ER stressor (FIG. 3A). We also determined that the sensitivity to ER stress in Wolfram cells was not cell line dependent, or dependent on the method used to generate iPS cells. A reduction in stimulated insulin secretion was observed for beta cells generated from all four Wolfram subjects, but not for a carrier and another control iPSC line (FIG. 3B). The reduced beta cell function was seen with iPS cells independent of the method of generation (FIGS. 7A and 7B) and also did not depend on the ER stressor: a reduction in insulin secretion was also observed in tunicamycin (TM)-treated Wolfram beta cells upon potassium stimulation (FIG. 8).

To determine whether the decreased responsiveness to secretagogues might be related to insulin processing/packaging, we determined the ratio of proinsulin/insulin in beta cells (FIG. 3C). We found that the proinsulin/insulin ratio in Wolfram beta cells was ˜0.55, similar to control cells (˜0.47). However, when cells were challenged with TG, the proinsulin to insulin ratio in the Wolfram beta cells increased to 0.73, which was significantly higher than that in control beta cells (0.51, P=0.03). 4PBA treatment restored normal insulin processing in TG-exposed Wolfram beta cells.

Because of the specific expression of WFS1 in beta cells (FIG. 1E), but not in glucagon expressing cells, we would expect that mutations differentially affect beta cells and alpha cells. We differentiated Wolfram cells into clusters containing both glucagon expressing and insulin expressing cells (FIG. 1E) and stimulated these cells with arginine. As arginine stimulates both endocrine cell types, we were able to determine stimulated hormone secretion in the same experiment, with and without TG treatment. TG treatment reduced stimulated glucagon secretion in control and WFS1 cells by 28% and 24% respectively. In contrast, the reduction of stimulated insulin secretion only occurred in WFS1 mutant cells (−3% versus 43%) (FIG. 3D).

### Example 6

Declining Stimulated Insulin Secretion of Wolfram Beta Cells In Vivo

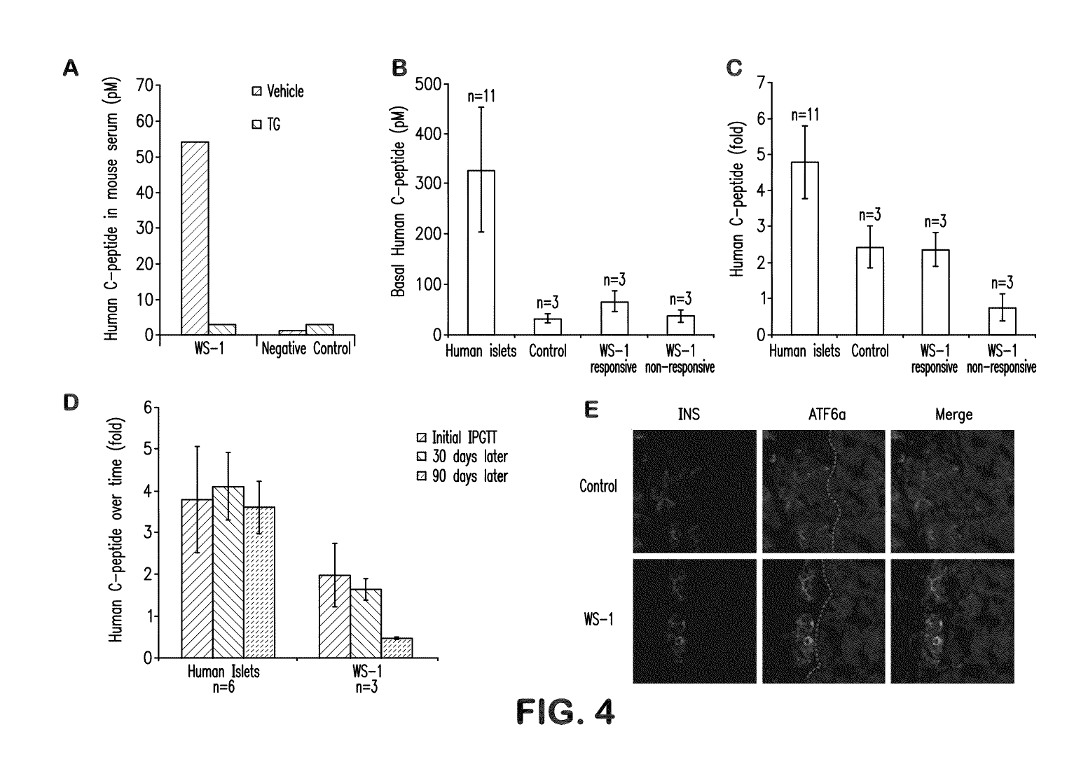

A potential limitation of an in vitro model is that it may not fully recapitulate all relevant characteristics due to the lack of a physiological (in vivo) environment that allows functional testing over a longer time period. After 14 days of in vitro differentiation, 2-3 million pancreatic endodermal cells were transplanted into the kidney capsule of immune-deficient mice. Human C-peptide was first detected 13 weeks post transplantation in the serum of mice transplanted with Wolfram and control cells in all, (6/6) mice. C-peptide originated from the graft, as human C-peptide became undetectable 2 days after the removal of the kidney containing the transplanted cells (FIG. 4A). All mice with Wolfram grafts had basal serum human C-peptide concentrations comparable to the control group (FIG. 4B). To determine the functional capacity of these grafts, intraperitoneal glucose tolerance tests (IPGTT) were performed. In 11 mice transplanted with human islets, C-peptide concentrations increased on average 4.78-fold (1.06-11.28 fold). Mice transplanted with control HUES-derived cells (n=3) showed a mean 2.43-fold increase (1.75-2.87 fold) of human C-peptide in serum. Mice transplanted with Wolfram-derived cells exhibited heterogeneous responses: 3 out of 6 mice showed a mean 2.35-fold increase of human C-peptide serum concentration, and the other 3 had no response to glucose (averaging a 0.75-fold reduction of human C-peptide) (FIG. 4C). Notably, grafts of Wolfram-derived cells, but not human islet controls lost their ability to respond to glucose within 90 days after the initial IPGTT test; fold induction remained 3.60 fold for human islets, and decreased below 1 for the Wolfram cells (FIG. 4D). Interestingly, although Wolfram implants lost their response to glucose, their basal secretion of human C-peptide remained stable (Initial average basal C-peptide was 58.18 pM, 30 days after was 55.71 pM and 90 days after was 95.44 pM). To determine the cause of impaired glucose-stimulated insulin secretion in Wolfram implants, one control and one Wolfram graft was isolated for histological analysis for the beta cell clusters. Although the insulin staining intensity of the Wolfram beta cells appeared similar to controls, a higher expression of ER stress marker, ATF6α was observed in transplanted graft containing Wolfram cells compared to control cells (FIG. 4E).

### Example 7

Results

A Stem Cell Model of ER Stress Induced Diabetes

Here we report a stem-cell based model of Wolfram syndrome, a fatal disorder characterized by diabetes with selective beta cell loss in the pancreas, as well as severe neuropathic phenotypes. Our model is remarkably faithful in recapitulating the beta cell physiology, and associated phenotypes seen in Wolfram syndrome. We found specific expression of WFS1 in beta cells and functional phenotypes ranging from reduced insulin content at low levels of ER stress, to a dilated endoplasmic reticulum, defective insulin processing, and a failure to secrete insulin in response to canonical stimuli at elevated levels of ER stress. Specific expression of WFS1 in beta cells has also been observed in mouse and human islets, and the phenotypes described are consistent with those reported in the mouse. For instance, a similar dilation of the ER and elevated ER stress markers have also been observed in a Wfs1 mutant mouse.

Despite the availability of a Wfs1 mutant mouse, the mechanisms how Wolframin mutations result in beta cell dysfunction and diabetes have remained unclear. Several models have been proposed for the role of WFS1 in beta cells, including generation of cAMP upon glucose stimulation, calcium homeostasis in the ER, a role in insulin processing and or as a negative regulator of the unfolded protein response by inhibiting ATF6 induced transcription. Our results are consistent with a primary role of WFS1 in protecting beta cells from protein folding stress and ER dysfunction. Beta cells of control subjects were resistant to experimentally induced ER stress, but rapidly lost functionality in the absence of WFS1. At the same concentrations of ER stress effectors, glucagon producing alpha cells of both control and wolfram mutant genotypes were affected to an equal and smaller extent than beta cells. We and others found that all three major pathways of UPR signaling are activated in the absence of WFS1, including PERK, IRE1 and ATF6, suggests that WFS1 primarily acts upstream of UPR signaling and not by regulating the activity of a particular UPR pathway. Under normal physiological conditions, the absence of WFS1 in beta cells results in elevated UPR signaling and a reduction of insulin synthesis. A further increase in ER stress causes beta cell failure by affecting insulin processing and stimulated insulin secretion. These phenotypes observed in vitro likely reflect beta cell failure after transplantation in vivo: glucose stimulated insulin secretion was initially present in some of the mice transplanted with human Wolfram cells, but over a time period of 90 days, the ability to increase insulin secretion in response to glucose was lost, and ER stress markers were increased in comparison to controls.

Stem Cell Model to Identify Compounds that Protect Beta Cells and Enhance their Function

Our model of Wolfram syndrome provides a platform for drug discovery and testing. We found that the chemical chaperone 4PBA is effective at reverting ER stress associated phenotypes in beta cells. This molecule or compounds with similar activity may be useful in preventing or delaying beta-cell dysfunction in Wolfram syndrome, and possibly other forms of diabetes.

Our results using Wolfram syndrome cells show that these cells reflect the phenotype of the affected subject. In addition to being relevant for Wolfram syndrome, our observations are likely relevant for other forms of diabetes. Unresolved ER stress may result in an inability of beta cells to secrete insulin in response to nutrients, and eventually beta cell death in all forms of diabetes. Beta cells of T2D and T1D subjects may have greater intrinsic ability to increase insulin synthesis in response to metabolic demand than Wolfram cells, but likely encounter a similar mismatch between metabolic demand and the ability to increase insulin production, resulting in elevated UPR signaling. In T1D, a decreasing number of beta cells endeavor to meet metabolic demand for insulin, and in most instances of T2D, the demand for insulin is increased because of peripheral insulin resistance. Increased expression of ER stress marker genes has been observed in the islets of type I diabetic mice and humans. Activation of ER stress associated genes (i.e. PERK and GRP78) has also been observed in the liver of mouse models of T2D and a higher susceptibility to ER stress induced by metabolic perturbations was observed in isolated islets in T2D patients. Reducing the demand for insulin by intensive insulin therapy improves endogenous beta cell function in T1D, and improving insulin sensitivity by PPARg inhibitors or by weight loss meliorates T2D, in part because beta cell function is improved. Common alleles in WFS1 are associated with increased diabetes risk. In the aggregate these earlier studies and those reported here support the concept of a role for ER stress in mediating aspects of the susceptibility and response of beta cells to failure in the context of diabetes.

Stem cell models of diabetes can be used for drug discovery and drug screening. We have identified two drugs, 4-PBA and TUDCA that reduce the activity of ER stress pathways, and improve beta cell function in a stem cell model of Wolfram syndrome. Our results suggest that the most effective intervention to restore some beta cell function in diabetes would be to reduce the demand for insulin (reduce the requirement for insulin synthesis), and at the same time to facilitate protein folding using chemical chaperones to reduce endoplasmic reticulum stress.

Although the invention has been described with reference to the above examples, it will be understood that modifications and variations are encompassed within the spirit and scope of the invention. Accordingly, the invention is limited only by the following claims.

We claim:

1. A method of treating a disease or disorder in a subject, wherein the disease or disorder is characterized by intracellular endoplasmic reticulum (ER) stress, comprising administering to the subject, an effective amount of a compound that is an ER stress reliever, thereby treating the disease or disorder.

2. The method of claim 1, wherein the compound is 4-phenylbutyric acid (PBA) or Tauroursodeoxycholic acid (TUDCA).

3. The method of claim 1, wherein the disease or disorder is diabetes.

4. The method of claim 1, wherein the disease or disorder is selected from the group consisting of Wolcott-Rallison syndrome, Permanent neonatal Diabetes, PERK−/− (global elevation or ER stress) and Wolfram syndrome.

5. A method of inhibiting beta cell loss in a subject with diabetes, comprising administering to the subject, an effective amount of an ER stress reliever compound, thereby inhibiting beta cell loss in the subject.

6. The method of claim 5, wherein the compound is 4-phenylbutyric Acid (PBA) or Tauroursodeoxychlic Acid (TUDCA).

7. The method of claim 1 or 5, further administering insulin to the subject.

8. The method of claim 1 or 5, wherein the subject is a mammal.

9. The method of claim 1 or 5, wherein the subject is a human.

10. The method of claim 3 or 5, wherein the subject has Type 1 or Type 2 diabetes.

11. The method of claim 1 or 5, wherein the compound is administered orally, rectally, transdermally, subcutaneously, intraveneously, intramuscularly, intrathecally, intraperiodontally, or intranasaly.

12. A method of identifying a compound that is an ER stress reliever comprising contacting a beta cell, in vitro or in vivo, with a test compound and measuring the level of insulin produced or protein folding prior to and following contacting with the test compound, wherein an increase in insulin levels or alteration in protein folding after contacting is indicative of an ER stress reliever compound.

13. The method of claim 12, wherein the beta cell is derived from a subject having diabetes.

14. The method of claim 12, wherein the beta cell is derived from a pluripotent stem cell of a subject having diabetes.

15. The method of claim 14, wherein the pluripotent stem cell is an iPSC.

16. The method of claim 1 or 5, wherein the compound is a chemical chaperone.

17. The method of claim 1 or 5, wherein the compound is a small molecule.


SEQUENCE LISTING (8 sequences)

SEQ ID NO: 1 - DNA, 20 bp, Artificial Sequence
  Primer
gaagccaaggggaatgaagt

SEQ ID NO: 2 - DNA, 20 bp, Artificial Sequence
  Primer
gggaagggcatttgaagaac

SEQ ID NO: 3 - DNA, 18 bp, Artificial Sequence
  Primer
ctgagtccgcagcaggtg

SEQ ID NO: 4 - DNA, 21 bp, Artificial Sequence
  Primer
tgcccaacaggatatcagact

SEQ ID NO: 5 - DNA, 20 bp, Artificial Sequence
  Primer
cacagtggtgcctaccaaga

SEQ ID NO: 6 - DNA, 21 bp, Artificial Sequence
  Primer
tgattgtcttttgtcaggggt

SEQ ID NO: 7 - DNA, 20 bp, Artificial Sequence
  Primer
ttctacacacccaagacccg

SEQ ID NO: 8 - DNA, 17 bp, Artificial Sequence
  Primer
caatgccacgcttctgc